(19) United States
(12) Patent Application Publication
(10) Pub. No.: US 2015/0278623 A1
(43) Pub. Date: Oct. 1, 2015

(54) SYSTEMS AND METHODS FOR PREVENTING WRONG-LEVEL SPINAL SURGERY

(71) Applicant: Blue Belt Technologies, Inc., Plymouth, MN (US)

(72) Inventor: Constantinos Nikou, Monroeville, PA (US)

(21) Appl. No.: 14/670,673

(22) Filed: Mar. 27, 2015

Related U.S. Application Data
(60) Provisional application No. 61/971,295, filed on Mar. 27, 2014.

(51) Int. Cl. G06K 9/20; A61B 19/00; G06T 11/00

(52) U.S. Cl. CPC G06K 9/2063; G06T 11/005; A61B 19/56; A61B 19/50; G06T 2200/24; G06T 2207/10081; G06T 2207/20101; G06T 2207/20104; G06T 2207/30012; G06T 2207/30196; G06T 2207/30204; G06T 2210/41; G06T 2211/428; G06T 2211/416; A61B 2019/566; A61B 2019/507

(57) ABSTRACT

Embodiments of a system and method for preventing wrong-level spinal surgery are generally described herein. A system can include a receiver to: receive an indication of a landmark on a patient corresponding to a single landmark point on an image of the patient, and receive an initial physical position of a tracked device in relation to the landmark on the patient. The system can include a processor to register the initial physical position of the tracked device to the landmark point on the image based on receiving the indication of the landmark on the patient, and a user interface to: display a first virtual position indicator at the landmark point on the image, and display, in response to the receiver receiving information indicating movement of the tracked device, a second virtual position indicator to indicate a linear movement of the second virtual position indicator in reference to the image.

### CLAIM OF PRIORITY

This patent application claims the benefit of priority of U.S. Provisional Patent Application Ser. No. 61/971,295, titled “Systems and Methods for Preventing Wrong-Level Spinal Surgery,” filed on Mar. 27, 2014, which is hereby incorporated by reference herein in its entirety.

### TECHNICAL FIELD

This document relates generally, but not by way of limitation, to systems and methods in the field of computer-aided spinal surgery.

### BACKGROUND

Due to the bilateral symmetry of the human body, it is a well-known problem in orthopedics that surgeons sometimes erroneously perform a procedure on the incorrect side of the body of a patient. For example, a patient is admitted for ACL reconstruction on the left knee, but the surgery is instead performed on the right knee. To combat this problem, methods and checklists have been developed that provide additional confirmatory information to the surgical team, which have achieved varying degrees of success.

A different, but related problem, arises in the area of spinal surgery because, even for skilled technicians, human error can still occur in identifying the area of the spine or vertebra on an actual patient's body that corresponds with a problem identified on a diagnostic image. This is due not only to variations in image quality and the skill or experience of the practitioner, but also to different body types and anatomical variability from one person to the next. This problem is compounded by the advent of minimally invasive surgery because the practitioner is actually able to see fewer landmarks or other internal structures than in open surgery. Thus, there are fewer data points or indications to either confirm or refute the selection of a particular vertebral body.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 illustrates generally a system including a tracked probe and a user interface for use on a patient in accordance with some embodiments.

FIG. 2 illustrates generally a schematic drawing showing a system for using a tracked device in accordance with some embodiments.

FIGS. 3A-3C illustrate generally tracked devices in accordance with some embodiments.

FIG. 4 illustrates generally, a user interface for displaying a virtual patient and a landmark point in accordance with some embodiments.

FIG. 5 illustrates generally an example of a block diagram of a machine upon which any one or more of the techniques (e.g., methodologies) discussed herein may perform in accordance with some embodiments.

FIG. 6 illustrates generally a method of identifying a desired surgical site on a patient's body in accordance with some embodiments.

### DETAILED DESCRIPTION

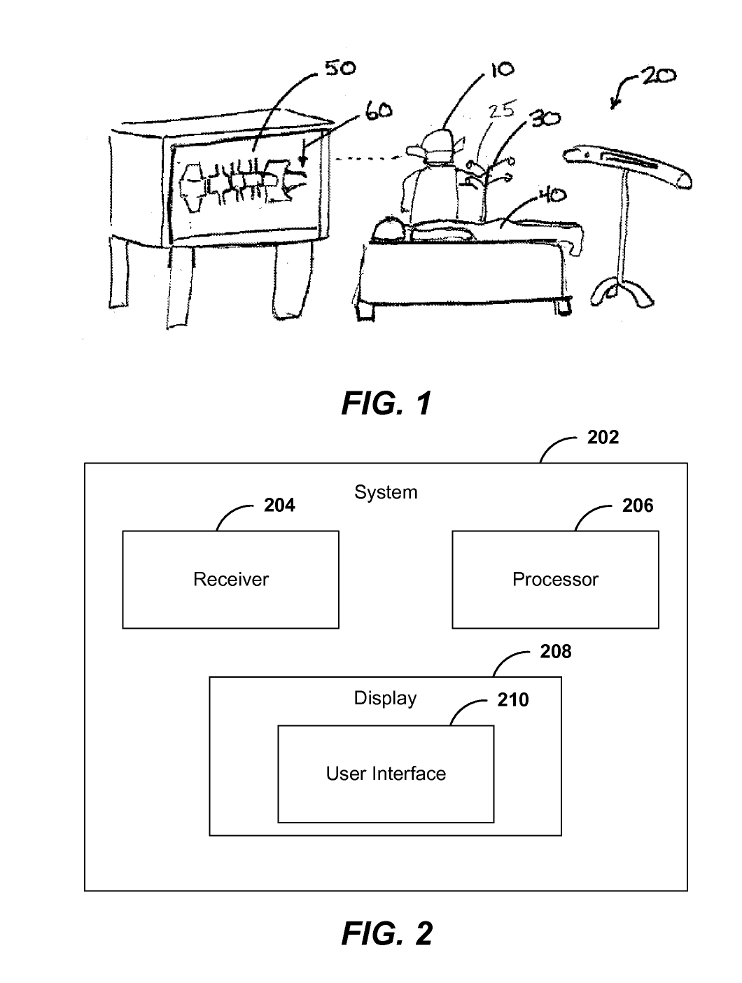

FIG. 1 illustrates generally a system including a tracked probe 30 and a user interface for use on a patient 40 in accordance with some embodiments. Referring to FIG. 1, a member of a surgical team 10 uses an optical tracking system 20 employed with surgical tools that have infrared reflector arrays 25 affixed thereto, as is known in the art. A more complete description of this well-known tracking system is set forth in U.S. Pat. No. 6,757,582 to Brisson, et. al., the entirety of which is hereby incorporated by reference as if more fully set forth herein.

In accord with an embodiment of the invention, the member of the surgical team 10 uses a tracked probe 30 to register one or more well-known landmarks, such as the coccyx, between the patient 40 and the digital images 50 that have been pre-imported into a computer running the tracking system as previously described. Once this landmark location data is collected, each data point is correlated to the same landmark location in the images 50 of the patient's anatomy so that the proportional difference between the image or images and the movement of the tracked probe is known. When the tracked probe 30 is moved linearly along the patient's spine, the software is configured to represent the actual location 60 of the probe on the digital image in order to ensure the surgeon marks the correct vertebral level before making an incision.

In an alternative embodiment, a tracked device having an extendable filament or tape is used. In use, the surgical team member can hold one end of the filament at a known landmark and extend or unwind the filament in much the same way a tape measure is extended or unwound. An encoder on the device can track the amount of tape or filament extended and transmit that information to the system where it can convert that linear distance to the scale of a relevant diagnostic image.

In still another alternative embodiment, an encoder can be employed to monitor the number of revolutions that a wheel on the tracked device travels when it is run linearly along the patient's back from a landmark point until the wheel reaches the appropriate vertebrae as represented or superimposed on the diagnostic image. This can be similar to the survey wheel encoders used by surveyors, but on a smaller scale.

In yet another embodiment, sound is employed in conjunction with the system to alert the surgical team when the tracked tool is close to or directly on top of the desired vertebrae. In this embodiment, the surgical team would not need to look away from the patient to know where the correct surgical site will be.

FIG. 2 illustrates generally a schematic drawing showing a system 202 for using a tracked device in accordance with some embodiments. The system 202 can include a receiver 204, a processor 206, and a display 208. The display 208 can include a user interface 210. The components of the system 202 can be integrated into one or more devices, including entirely in one device or entirely separately. In an embodiment, the receiver 204 can receive an indication of a landmark on a patient (e.g., the coccyx, or other easily identified landmark), wherein the landmark corresponds to a single landmark point on an image of the patient. The landmark on the patient can be identified pre-operatively or inter-operatively, such as through the skin without an incision or penetration. The receiver 204 can further receive an initial physical position of a tracked device in relation to the landmark on the patient. For example, the physical position of the tracked device can be determined using a local positioning system or other technique for determining an absolute or relative position of the tracked device, which can then be received by the receiver 204.

In an alternative embodiment, the receiver 204 can receive information indicating extension of an extensible tracked device starting at the first landmark on the patient. In yet another embodiment, the receiver 204 can receive an indication of a tracked device at a first landmark of a patient. For example, the receiver 204 can receive an indication of an initialization of the tracked device, and can do so without receiving any position data from the tracked device. In such an example, the system operates under the assumption that the member of the surgical team started the tracked device at a position on the patient that coincides with the landmark determined pre-operatively on the diagnostic image.

The processor 206 can register an initial physical position of the tracked device (when the receiver 204 receives the initial physical position), such as by registering the position to a landmark point on the image. In another embodiment, the processor 206 can register an indication of a first landmark on a patient to a first landmark point on an image. The processor 206 can receive an initialization indication from the tracked device at the first landmark on the patient. The processor 206 can receive the initialization indication from the receiver 204, which can receive the initialization indication from the tracked device.

The display 208 can be a physical display, such as a monitor or television screen. The user interface 210 displays an image of a patient, such as an image of a bone (e.g., a spine). The user interface 210 can display a reference point, a landmark point, and one or more virtual position indicators. The features of the user interface 210 are described in more detail below.

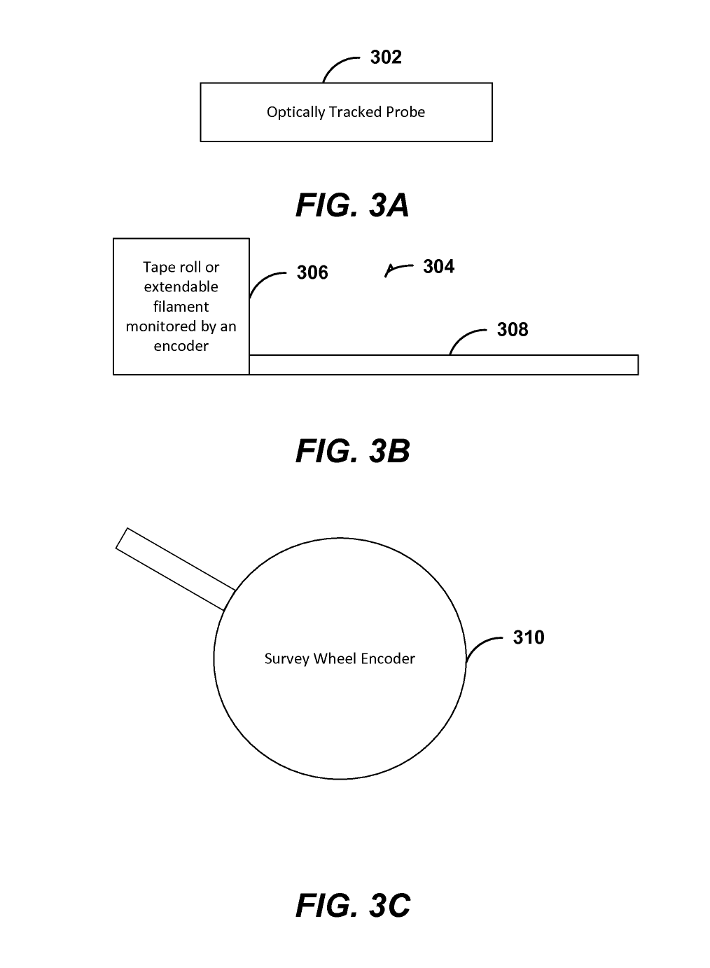

FIGS. 3A-3C illustrate generally tracked devices in accordance with some embodiments. The tracked devices can include a transceiver or a cable to communicate with a remote device, such as the receiver 204 of FIG. 2. The tracked devices can be configured to traverse a patient from a first landmark on the patient to a second landmark (or second position along the anatomy of interest, such as the spine) on the patient. The tracked devices can move linearly from a first landmark on a patient along a bone, such as a spine. FIG. 3A illustrates an optically tracked probe 302. For example, the optically tracked probe 302 can include the optical tracking system 20 of FIG. 1 with the infrared reflector arrays 25.

FIG. 3B illustrates a tape roll or extendable filament monitored by an encoder 304. For example, a tape roll an extendable filament monitored by an encoder 304 can include a main portion 306 and an extension portion 308. The main portion 306 can include an encoder to measure a current extension length of the extension portion 308. FIG. 3C illustrates a survey wheel encoder 310. The survey wheel encoder 310 can include an encoder to measure a change in location or a distance. The tape roll or extendable filament monitored by an encoder 306 or the survey wheel encoder 310 can include an extensible tracked device.

In another embodiment, movement or a change in location or distance of a tracked device can include movement along skin of a patient. For example, the distance traveled by the survey wheel encoder 310 as it moves along the skin of a patient can be determined by the encoder and can be transmitted to a user interface. The user interface can automatically trace a skin contour on an image corresponding to the movement of the survey wheel encoder 310 along the skin of the patient. Similarly, a user interface can receive information from the tape roll or extendable filament monitored by an encoder 306 about the length of an extension portion extended along a patient, such as flush with the skin of the patient. Other embodiments can use curve distances instead of or in combination with linear distances to automatically update a virtual position indicator on a user interface.

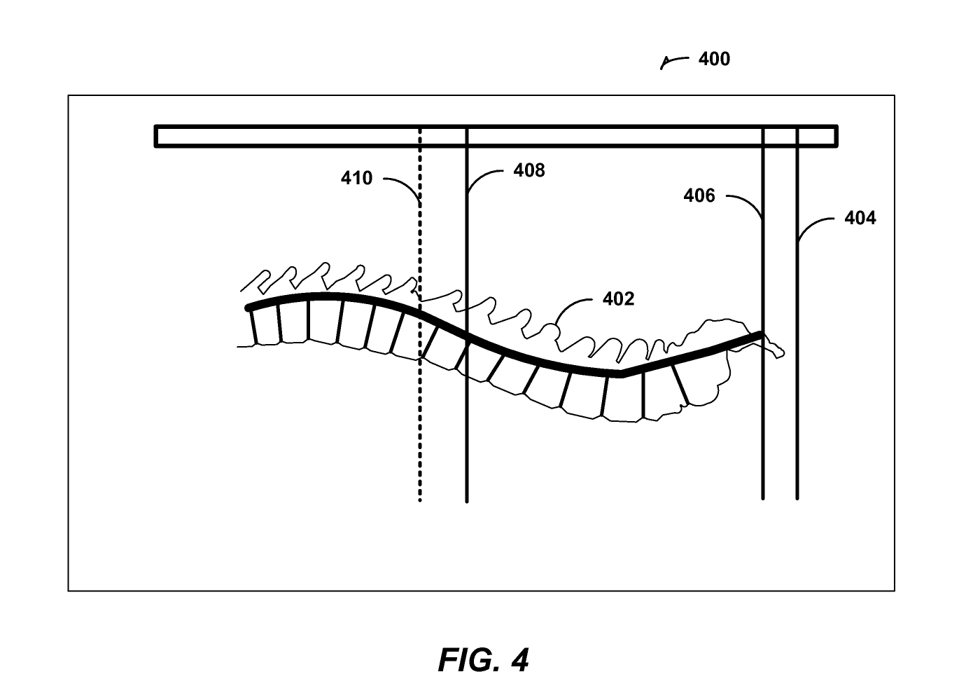

FIG. 4 illustrates generally, a user interface 400 for displaying a virtual bone 402 of a patient and a landmark point 406 in accordance with some embodiments. The user interface 400 can include the virtual bone 402, the landmark point 406, a reference point 404, a virtual position indicator 408 and a virtual representation of a desired surgical site 410. The virtual bone 402 can include a spine. The reference point 404 can be predetermined from an image taken of a patient. The image can include a slice, cut, or collage of a diagnostic image taken of a patient, such as a CT or CAT scan or an MRI. The virtual bone 402 is generated from the diagnostic image through various technique known in the art. The image can include known dimensions for the virtual bone 402 that can be translated to distances on the patient. The image can include a portion of anatomy of a patient at a known scale. The reference point 404 can be an edge of the image.

The user interface 400 can be used to identify the landmark point 406. For example, a user can identify the landmark point 406 by touching the user interface, moving a pointer device (e.g., mouse), or entering an axial position using a keyboard or other input. In another embodiment, the landmark point 406 can be automatically determined. The landmark point 406 can be a first virtual position indicator, the virtual position indicator 408 can be a second virtual position indicator, and the virtual representation of the desired surgical site 410 can be a third virtual position indicator.

The virtual position indicator 408 can move along the bone 402 in the image on the user interface 400. For example, the virtual position indicator 408 can move from the landmark point 406 or the reference point 404. The movement of the virtual position indicator 408 can automatically correspond to movement of a tracked probe (e.g., the tracked probes in FIGS. 3A-3C) or portions of a tracked probe (e.g., the extension portion 308 of FIGS. 3B-3C). As the virtual position indicator 408 moves from the landmark point 406 along the bone 402, it can reach the virtual representation of the desired surgical site 410. When the virtual position indicator 408 reaches the virtual representation of the desired surgical site 410, the user interface 400 can issue an alert, such as a flash on the user interface 400, an audible alert, or a haptic alert (e.g., a vibration of the tracked probe). The virtual position indicator 408 can reach the virtual representation of the desired surgical site 410 when the tracked probe reaches or is substantially adjacent to the desired surgical site on the patient. In another embodiment, the virtual position indicator 408 can move and reach the virtual representation of the desired surgical site 410 when an end of an extension portion of an extendable tracked device reaches the desired surgical site or an encoder of an extendable tracked device indicates that a specified portion of the extendable tracked device has reached the desired surgical site.

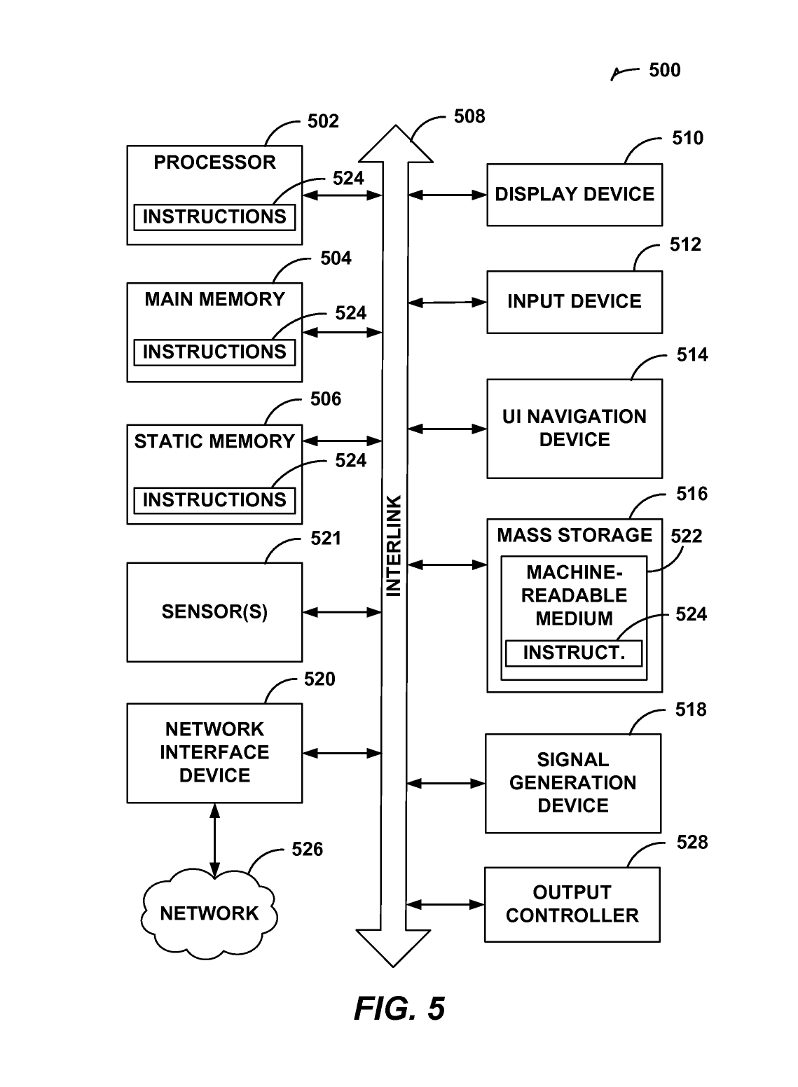

FIG. 5 illustrates generally an embodiment of a block diagram of a machine 500 upon which any one or more of the techniques (e.g., methodologies) discussed herein may perform in accordance with some embodiments. In alternative embodiments, the machine 500 may operate as a standalone device or may be connected (e.g., networked) to other machines. In a networked deployment, the machine 500 may operate in the capacity of a server machine, a client machine, or both in server-client network environments. In an embodiment, the machine 500 may act as a peer machine in peer-to-peer (P2P) (or other distributed) network environment. The machine 500 may be a personal computer (PC), a tablet PC, a set-top box (STB), a personal digital assistant (PDA), a mobile telephone, a web appliance, a network router, switch or bridge, or any machine capable of executing instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein, such as cloud computing, software as a service (SaaS), other computer cluster configurations.

Embodiments, as described herein, may include, or may operate on, logic or a number of components, modules, or mechanisms. Modules are tangible entities (e.g., hardware) capable of performing specified operations when operating. A module includes hardware. In an embodiment, the hardware may be specifically configured to carry out a specific operation (e.g., hardwired). In an embodiment, the hardware may include configurable execution units (e.g., transistors, circuits, etc.) and a computer readable medium containing instructions, where the instructions configure the execution units to carry out a specific operation when in operation. The configuring may occur under the direction of the executions units or a loading mechanism. Accordingly, the execution units are communicatively coupled to the computer readable medium when the device is operating. In this embodiment, the execution units may be a member of more than one module. For example, under operation, the execution units may be configured by a first set of instructions to implement a first module at one point in time and reconfigured by a second set of instructions to implement a second module.

Machine (e.g., computer system) 500 may include a hardware processor 502 (e.g., a central processing unit (CPU), a graphics processing unit (GPU), a hardware processor core, or any combination thereof), a main memory 504 and a static memory 506, some or all of which may communicate with each other via an interlink (e.g., bus) 508. The machine 500 may further include a display unit 510, an alphanumeric input device 512 (e.g., a keyboard), and a user interface (UI) navigation device 514 (e.g., a mouse). In an embodiment, the display unit 510, alphanumeric input device 512 and UI navigation device 514 may be a touch screen display. The machine 500 may additionally include a storage device (e.g., drive unit) 516, a signal generation device 518 (e.g., a speaker), a network interface device 520, and one or more sensors 521, such as a global positioning system (GPS) sensor, compass, accelerometer, or other sensor. The machine 500 may include an output controller 528, such as a serial (e.g., universal serial bus (USB), parallel, or other wired or wireless (e.g., infrared (IR), near field communication (NFC), etc.) connection to communicate or control one or more peripheral devices (e.g., a printer, card reader, etc.).

The storage device 516 may include a machine readable medium 522 that is non-transitory on which is stored one or more sets of data structures or instructions 524 (e.g., software) embodying or utilized by any one or more of the techniques or functions described herein. The instructions 524 may also reside, completely or at least partially, within the main memory 504, within static memory 506, or within the hardware processor 502 during execution thereof by the machine 500. In an embodiment, one or any combination of the hardware processor 502, the main memory 504, the static memory 506, or the storage device 516 may constitute machine readable media.

While the machine readable medium 522 is illustrated as a single medium, the term “machine readable medium” may include a single medium or multiple media (e.g., a centralized or distributed database, and/or associated caches and servers) configured to store the one or more instructions 524.

The term “machine readable medium” may include any medium that is capable of storing, encoding, or carrying instructions for execution by the machine 500 and that cause the machine 500 to perform any one or more of the techniques of the present disclosure, or that is capable of storing, encoding or carrying data structures used by or associated with such instructions. Non-limiting machine readable medium examples may include solid-state memories, and optical and magnetic media. In an embodiment, a massed machine readable medium comprises a machine readable medium with a plurality of particles having invariant (e.g., rest) mass. Accordingly, massed machine-readable media are not transitory propagating signals. Specific examples of massed machine readable media may include: non-volatile memory, such as semiconductor memory devices (e.g., Electrically Programmable Read-Only Memory (EPROM), Electrically Erasable Programmable Read-Only Memory (EEPROM)) and flash memory devices; magnetic disks, such as internal hard disks and removable disks; magneto-optical disks; and CD-ROM and DVD-ROM disks.

The instructions 524 may further be transmitted or received over a communications network 526 using a transmission medium via the network interface device 520 utilizing any one of a number of transfer protocols (e.g., frame relay, internet protocol (IP), transmission control protocol (TCP), user datagram protocol (UDP), hypertext transfer protocol (HTTP), etc.). Example communication networks may include a local area network (LAN), a wide area network (WAN), a packet data network (e.g., the Internet), mobile telephone networks (e.g., cellular networks), Plain Old Telephone (POTS) networks, and wireless data networks (e.g., Institute of Electrical and Electronics Engineers (IEEE) 802.11 family of standards known as Wi-Fi®, IEEE 802.16 family of standards known as WiMax®), IEEE 802.15.4 family of standards, peer-to-peer (P2P) networks, among others. In an embodiment, the network interface device 520 may include one or more physical jacks (e.g., Ethernet, coaxial, or phone jacks) or one or more antennas to connect to the communications network 526. In an embodiment, the network interface device 520 may include a plurality of antennas to wirelessly communicate using at least one of single-input multiple-output (SIMO), multiple-input multiple-output (MIMO), or multiple-input single-output (MISO) techniques. The term “transmission medium” shall be taken to include any intangible medium that is capable of storing, encoding or carrying instructions for execution by the machine 500, and includes digital or analog communications signals or other intangible medium to facilitate communication of such software.

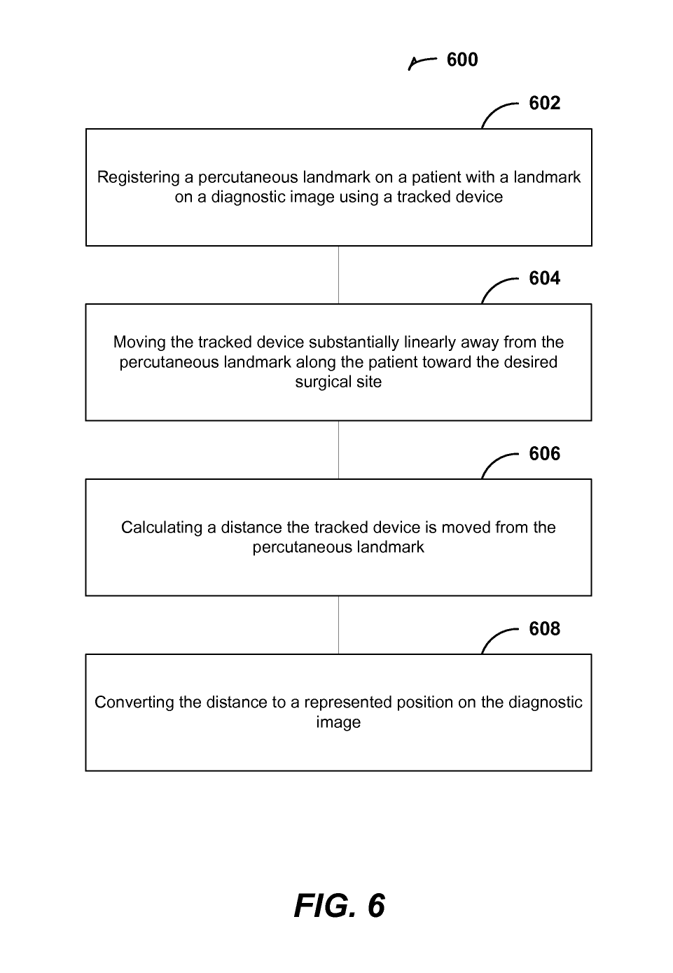

FIG. 6 illustrates generally a method 600 of identifying a desired surgical site on a patient's body in accordance with some embodiments. The method 600 can include an operation 602 to register a percutaneous landmark on a patient with a landmark on a diagnostic image using a tracked device. The method 600 can include an operation 604 to move the tracked device substantially linearly away from the percutaneous landmark along the patient toward the desired surgical site. The method 600 can include an operation 606 to calculate a distance the tracked device is moved from the percutaneous landmark. The method 600 can include an operation 608 to convert the distance to a represented position on the diagnostic image. The method 600 can further comprise providing confirmation when the tracked device is substantially adjacent to the desired surgical site. The landmark can include a point on a bone of the patient, such as a coccyx and the desired surgical site can include a vertebra. The tracked device can include a survey wheel encoder, an extendable filament monitored by an encoder, a tape roll, or a tracked probe.

### VARIOUS NOTES & EXAMPLES

Each of these non-limiting examples can stand on its own, or can be combined in various permutations or combinations with one or more of the other examples.

Example 1 includes the subject matter embodied by a system comprising: a receiver to: receive an indication of a landmark on a patient corresponding to a single landmark point on an image of the patient, and receive an initial physical position of a tracked device in relation to the landmark on the patient, a processor to register the initial physical position of the tracked device to the landmark point on the image based on receiving the indication of the landmark on the patient, a user interface to: display a first virtual position indicator at the landmark point on the image, and display, in response to the receiver receiving information indicating movement of the tracked device, a second virtual position indicator to indicate a linear movement of the second virtual position indicator in reference to the image.

In Example 2, the subject matter of Example 1 can optionally include wherein the receiver is further to receive an indication of the single landmark point on the image of the patient.

In Example 3, the subject matter of one or any combination of Examples 1-2 can optionally include wherein to receive the physical position of the tracked device, the receiver is to receive the physical position of the tracked device after initialization of the tracked device.

In Example 4, the subject matter of one or any combination of Examples 1-3 can optionally include wherein the image of the patient includes an image of a spine of the patient.

In Example 5, the subject matter of one or any combination of Examples 1-4 can optionally include wherein the image includes a portion of a CT scan.

In Example 6, the subject matter of one or any combination of Examples 1-5 can optionally include wherein the tracked device includes one of a survey wheel encoder, a tape roll, an extendable filament monitored by an encoder, or an optically tracked probe.

In Example 7, the subject matter of one or any combination of Examples 1-6 can optionally include wherein the user interface is further to display a third virtual position indicator on the image at a location corresponding to a desired surgical site on the patient.

In Example 8, the subject matter of one or any combination of Examples 1-7 can optionally include wherein the user interface is further to provide confirmation when the tracked device is substantially adjacent to the desired surgical site.

Example 9 includes the subject matter embodied by a system comprising: a receiver to: receive an indication of a first landmark on a patient corresponding to a first landmark point on an image of the patient, receive information indicating extension of an extensible tracked device starting at the first landmark on the patient, a processor to register the indication of the first landmark on the patient to the first landmark point on the image, a user interface to: display a first virtual position indicator at the first landmark point on the image, and display, in response to the receiver receiving the information indicating extension of the extensible tracked device to a second landmark on the patient, a second virtual position indicator at a second landmark point on the image, the second landmark point on the image corresponding to the second landmark on the patient.

In Example 10, the subject matter of Example 9 can optionally include wherein the extensible tracked device includes one of a survey wheel encoder, a digital tape measure, or an extendable filament monitored by an encoder.

Example 11 includes the subject matter embodied by a system comprising: a user interface configured to display a first virtual position indicator at a first landmark point on an image of a patient, a tracked device configured to traverse a patient from a first landmark on the patient to a second landmark on the patient, a processor configured to: register the first landmark on the patient to the first landmark point on the image based on receiving an indication of the first landmark on the patient, wherein the user interface is further configured to display a second virtual position indicator at a second landmark point on the image, the second landmark point corresponding to the second landmark on the patient.

In Example 12, the subject matter of Example 11 can optionally include wherein the processor is configured to receive an initialization indication from the tracked device at the first landmark on the patient.

In Example 13, the subject matter of one or any combination of Examples 11-12 can optionally include wherein the user interface is configured to display the first virtual position indicator at the first landmark point after the system receives a user indication of the first landmark point on the image.

In Example 14, the subject matter of one or any combination of Examples 11-13 can optionally include wherein the image includes a portion of anatomy of the patient at a known scale.

In Example 15, the subject matter of one or any combination of Examples 11-14 can optionally include wherein the tracked device includes one of a survey wheel encoder, a tape roll, an extendable filament monitored by an encoder, or an optically tracked probe.

In Example 16, the subject matter of one or any combination of Examples 11-15 can optionally include wherein the user interface is further to display a third virtual position indicator on the image at a third landmark point corresponding to a desired surgical site on the patient.

In Example 17, the subject matter of one or any combination of Examples 11-16 can optionally include wherein the user interface is further configured to provide confirmation when the tracked device is substantially adjacent to the desired surgical site.

Example 18 includes the subject matter embodied by a machine-readable medium including instructions of receiving information, which when executed by a machine, cause the machine to: receive an indication of a landmark point on an image of a patient, display a first virtual position indicator at the landmark point on the image, receive an indication of a tracked device at a first landmark of a patient, register the first landmark to a first landmark point on the image, receive an indication of linear movement of the tracked device to a second landmark of the patient, display, a second virtual position indicator to indicate linear movement of the second virtual position indicator from the first landmark point on the image to a second landmark point on the image, the second landmark point corresponding to the second landmark of the patient, and wherein the linear movement of the second virtual position indicator corresponds to the linear movement of the tracked device.

In Example 19, the subject matter of Example 18 can optionally include wherein the image includes a portion of a CT scan.

In Example 20, the subject matter of one or any combination of Examples 18-19 can optionally include wherein the tracked device includes one of a survey wheel encoder, a digital tape measure, an extendable filament monitored by an encoder, or an optically tracked probe.

In Example 21, the subject matter of one or any combination of Examples 18-20 can optionally include operations to display a third virtual position indicator on the image at a third virtual position indicator on the image at a third landmark point corresponding to a desired surgical site on the patient.

In Example 22, the subject matter of one or any combination of Examples 18-21 can optionally include operations to provide confirmation when the tracked device is substantially adjacent to the desired surgical site.

In Example 23, the subject matter of one or any combination of Examples 18-22 can optionally include wherein the confirmation includes at least one of an audible confirmation, a visual confirmation, and a haptic confirmation.

Example 24 includes the subject matter embodied by a method of identifying a desired surgical site on a patient comprising: registering a percutaneous landmark on the patient with the landmark on a diagnostic image using a tracked device, moving the tracked device substantially linearly away from the percutaneous landmark along the patient toward the desired surgical site, calculating a distance the tracked device is moved from the percutaneous landmark, and converting the distance to a represented position on the diagnostic image.

In Example 25, the subject matter of Example 24 can optionally include providing confirmation when the tracked device is substantially adjacent to the desired surgical site.

In Example 26, the subject matter of one or any combination of Examples 24-25 can optionally include wherein the landmark is the patient's coccyx and the desired surgical site is a vertebra.

In Example 27, the subject matter of one or any combination of Examples 24-26 can optionally include wherein the tracked device comprises a survey wheel encoder.

In Example 28, the subject matter of one or any combination of Examples 24-27 can optionally include wherein the tracked device comprises an extendable filament monitored by an encoder.

In Example 29, the subject matter of one or any combination of Examples 24-28 can optionally include wherein the tracked device is a probe.

The above detailed description includes references to the accompanying drawings, which form a part of the detailed description. The drawings show, by way of illustration, specific embodiments. These embodiments are also referred to herein as “examples.” Such examples may include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein may be machine or computer-implemented at least in part. Some examples may include a computer-readable medium or machine-readable medium encoded with instructions operable to configure an electronic device to perform methods as described in the above examples. An implementation of such methods may include code, such as microcode, assembly language code, a higher-level language code, or the like. Such code may include computer readable instructions for performing various methods. The code may form portions of computer program products. Further, in an example, the code may be tangibly stored on one or more volatile, non-transitory, or non-volatile tangible computer-readable media, such as during execution or at other times. Examples of these tangible computer-readable media may include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments may be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments may be combined with each other in various combinations or permutations.

What is claimed is:

1. A system comprising:
a receiver to:
receive an indication of a landmark on a patient corresponding to a single landmark point on an image of the patient; and
receive an initial physical position of a tracked device in relation to the landmark on the patient;
a processor to register the initial physical position of the tracked device to the landmark point on the image based on receiving the indication of the landmark on the patient;
a user interface to:
display a first virtual position indicator at the landmark point on the image; and
display, in response to the receiver receiving information indicating movement of the tracked device, a second virtual position indicator to indicate a linear movement of the second virtual position indicator in reference to the image.

2. The system of claim 1, wherein the receiver is further to receive an indication of the single landmark point on the image of the patient.

3. The system of claim 1, wherein to receive the physical position of the tracked device, the receiver is to receive the physical position of the tracked device after initialization of the tracked device.

4. The system of claim 1, wherein the image of the patient includes an image of a spine of the patient.

5. The system of claim 1, wherein the image includes a portion of a CT scan.

6. The system of claim 1, wherein the tracked device includes one of a survey wheel encoder, a tape roll, an extendable filament monitored by an encoder, or an optically tracked probe.

7. The system of claim 1, wherein the user interface is further to display a third virtual position indicator on the image at a location corresponding to a desired surgical site on the patient.

8. The system of claim 7, wherein the user interface is further to provide confirmation when the tracked device is substantially adjacent to the desired surgical site.

9. A system comprising:
a receiver to:
receive an indication of a first landmark on a patient corresponding to a first landmark point on an image of the patient;
receive information indicating extension of an extensible tracked device starting at the first landmark on the patient;
a processor to register the indication of the first landmark on the patient to the first landmark point on the image;
a user interface to:
display a first virtual position indicator at the first landmark point on the image; and
display, in response to the receiver receiving the information indicating extension of the extensible tracked device to a second landmark on the patient, a second virtual position indicator at a second landmark point on the image, the second landmark point on the image corresponding to the second landmark on the patient.

10. The system of claim 9, wherein the extensible tracked device includes one of a survey wheel encoder, a digital tape measure, or an extendable filament monitored by an encoder.

11. A system comprising:
a user interface configured to display a first virtual position indicator at a first landmark point on an image of a patient;
a tracked device configured to traverse a patient from a first landmark on the patient to a second landmark on the patient;
a processor configured to:
register the first landmark on the patient to the first landmark point on the image based on receiving an indication of the first landmark on the patient;
wherein the user interface is further configured to display a second virtual position indicator at a second landmark point on the image, the second landmark point corresponding to the second landmark on the patient.

12. The system of claim 11, wherein the processor is configured to receive an initialization indication from the tracked device at the first landmark on the patient.

13. The system of claim 11, wherein the user interface is configured to display the first virtual position indicator at the first landmark point after the system receives a user indication of the first landmark point on the image.

14. The system of claim 11, wherein the image includes a portion of anatomy of the patient at a known scale.

15. The system of claim 11, wherein the tracked device includes one of a survey wheel encoder, a tape roll, an extendable filament monitored by an encoder, or an optically tracked probe.

16. The system of claim 11, wherein the user interface is further to display a third virtual position indicator on the image at a third landmark point corresponding to a desired surgical site on the patient.

17. The system of claim 11, wherein the user interface is further configured to provide confirmation when the tracked device is substantially adjacent to the desired surgical site.

18. A machine-readable medium including instructions of receiving information, which when executed by a machine, cause the machine to:
receive an indication of a landmark point on an image of a patient;
display a first virtual position indicator at the landmark point on the image;
receive an indication of a tracked device at a first landmark of a patient;
register the first landmark to a first landmark point on the image;
receive an indication of linear movement of the tracked device to a second landmark of the patient;
display, a second virtual position indicator to indicate linear movement of the second virtual position indicator from the first landmark point on the image to a second landmark point on the image, the second landmark point corresponding to the second landmark of the patient; and
wherein the linear movement of the second virtual position indicator corresponds to the linear movement of the tracked device.

19. The machine-readable medium of claim 18, wherein the image includes a portion of a CT scan.

20. The machine-readable medium of claim 18, wherein the tracked device includes one of a survey wheel encoder, a digital tape measure, an extendable filament monitored by an encoder, or an optically tracked probe.

21. The machine-readable medium of claim 18, further comprising operations to display a third virtual position indicator on the image at a third virtual position indicator on the image at a third landmark point corresponding to a desired surgical site on the patient.

22. The machine-readable medium of claim 21, further comprising operations to provide confirmation when the tracked device is substantially adjacent to the desired surgical site.

23. The machine-readable medium of claim 22, wherein the confirmation includes at least one of an audible confirmation, a visual confirmation, and a haptic confirmation.
